(19) United States
(12) Patent Application Publication
(10) Pub. No.: US 2025/0302286 A1
(43) Pub. Date: Oct. 2, 2025

(54) MEDICAL DEVICE ELECTRONICS ASSEMBLY

(71) Applicant: Boston Scientific Scimed, Inc., Maple Grove, MN (US)

(72) Inventors: Louis J. BARBATO, Franklin, MA (US); Eric F. BROWN, Wilmington, MA (US)

(73) Assignee: Boston Scientific Scimed, Inc., Maple Grove, MN (US)

(21) Appl. No.: 19/090,585

(22) Filed: Mar. 26, 2025

Related U.S. Application Data
(60) Provisional application No. 63/570,901, filed on Mar. 28, 2024.

(51) Int. Cl. A61B 1/05; A61B 1/00

(52) U.S. Cl. CPC A61B 1/051; A61B 1/00124

(57) ABSTRACT

Disclosed solutions relate to an electronic package for a medical device. In an example, the electronic package includes an electronic device with a rear device surface having a device electrical contact. The device further includes a printed circuit board (PCB) having a PCB surface with one or more electrical traces and an edge surface transverse to the PCB surface. The edge surface faces and is spaced from the rear device surface. The package further includes an electrical component fixed to the PCB surface and electrically connected with the device electrical contact and the one or more electrical traces.

### CROSS-REFERENCE TO RELATED APPLICATIONS

This application claims the benefit of priority under 35 U.S.C. § 119 from U.S. Provisional Application No. 63/570,901, filed on Mar. 28, 2024, which is incorporated by reference herein in its entirety.

### TECHNICAL FIELD

Various aspects of this disclosure relate generally to medical devices, for example scopes such as endoscopes or bronchoscopes, that include various electronic components. More specifically, aspects of this disclosure relate to electronic packages suitable for medical devices such as scopes.

### BACKGROUND

Endoscopes have attained great acceptance within the medical community as they provide a means for performing procedures with minimal patient trauma while enabling the physician to view the internal anatomy of the patient. Numerous endoscopes have been developed and categorized according to specific applications, such as cystoscopy, colonoscopy, bronchoscopy, upper GI endoscopy, and others.

An endoscope usually has an elongated tubular shaft, having a video camera or a fiber optic lens assembly at its distal end. The shaft is connected to a handle. Viewing is usually possible via an external display. Various surgical tools may be inserted through a working channel in the endoscope for performing different surgical procedures. Endoscopes, such as colonoscopes, typically have a front camera for viewing the internal organ, such as the colon, an illuminator, a fluid injector for cleaning the camera lens (and sometimes also the illuminator), and a working channel for insertion of surgical tools, for example, for removing polyps found in the colon. Often, endoscopes also have fluid injectors for cleaning a body cavity, such as the colon, into which they are inserted. The illuminators commonly used include fiber optics which transmit light to the endoscope tip section and light-emitting diodes (LEDs) at the endoscope tip section.

Current endoscopes typically implement an imaging system through a camera sensor at a tip section of the endoscope, such as a charge coupled device (CCD) sensor or a complementary metal-oxide semiconductor (CMOS) sensor. In forward looking endoscopes, a plane of the image sensor is often mounted orthogonal or approximately orthogonal, to the long axis of the endoscope.

But existing techniques for mounting an image sensor within a scope suffer deficiencies. For example, existing techniques can require bends that apply pressure on the image sensor, a circuit board, and/or associated wiring. Accordingly, there is a need in the art for improved electronic packages to facilitate mounting and connection of electronic devices such as image sensors within scopes.

### SUMMARY

Aspects of the disclosure relate to, among other things, systems, devices, and electronic packages for medical devices.

In some aspects, the embodiments disclosed herein relate to an electronic package for a medical device. The package includes an electronic device with a rear device surface having at least one device electrical contact. The package further includes a printed circuit board (PCB) having (i) a PCB surface with one or more electrical traces and (ii) an edge surface transverse to the PCB surface. The edge surface faces and is spaced from the rear device surface. The package further includes an electrical component fixed to the PCB surface and electrically connected with the at least one device electrical contact and the one or more electrical traces.

In some aspects, the electrical component is mounted flush with the edge surface.

In some aspects, the electrical component protrudes distally away from the edge surface.

In some aspects, the electrical component is mounted recessed from the edge surface.

In some aspects, the at least one device electrical contact protrudes from the rear device surface.

In some aspects, the printed circuit board is spaced from the at least one device electrical contact.

In some aspects, the electrical component includes at least one contact that is electrically connected with the at least one device electrical contact.

In some aspects, the printed circuit board further includes a bottom surface, an additional electrical component positioned on the bottom surface, and an additional contact positioned on the additional electrical component.

In some aspects, the additional electrical component is electrically connected to the electronic device.

In some aspects, the electronic device further includes a ball grid array (BGA) that includes the at least one device electrical contact.

In some aspects, the electrical component includes at least one electrical contact. The electronic device is an imaging sensor that is electrically connected, via the at least one device electrical contact and the at least one electrical contact, to the one or more electrical traces. In some aspects, the edge surface does not include any electrical traces or electrical contacts.

In some aspects, the printed circuit board includes a first arm and a second arm. The first arm and the second arm extend distally from a base of the printed circuit board. The electronic device is positioned between the first arm and the second arm and distally of the base. The printed circuit board includes an illumination source positioned at an end of either the first arm or the second arm.

In some aspects, the electrical component includes an insulator, a first component electrical contact, and a second component electrical contact spaced and insulated from the first component electrical contact, and the at least one device electrical contact includes a first device electrical contact and a second device electrical contact spaced and insulated from the first device electrical contact. The first component electrical contact touches the first device electrical contact, and the second component electrical contact touches the second device electrical contact.

In some aspects, the electrical component is a resistor or a capacitor.

In some aspects, the embodiments disclosed herein relate to an endoscope. The endoscope relates to an imaging sensor including a rear device surface having device electrical contacts. The imaging sensor is positioned in a distal end of the endoscope. The endoscope includes a printed circuit board (PCB) having a PCB surface and an edge surface transverse to the PCB surface. The edge surface faces the rear device surface and is spaced from the rear device surface. The endoscope includes an insulator affixed to the PCB surface. The endoscope includes electrical contacts affixed to the insulator and electrically connected to the device electrical contacts.

In some aspects, the printed circuit board includes a first arm and a second arm. The imaging sensor is positioned between the first arm and the second arm, the endoscope further including an illumination source positioned on either the first arm or the second arm.

In some aspects, the printed circuit board further includes one or more electrical traces. The imaging sensor is electrically connected, via the device electrical contacts and the electrical contacts, to the one or more electrical traces.

In some aspects, the embodiments disclosed herein relate to a connector for an electronic device. The connector includes a first arm and a second arm. The first arm and the second arm define a space therebetween configured to receive the electronic device. The connector includes a body portion at a base of the first arm and the second arm. The body portion includes a top planar surface with electrical traces; a bottom planar surface; an edge surface connecting the top planar surface and the bottom planar surface and facing the space; and an electrical contact that is disposed on the top planar surface. A contact surface of the electrical contact is either aligned with the edge surface or is above the space. The electrical contact is configured to electrically connect with a device contact of the electronic device.

In some aspects, the bottom planar surface includes an additional electrical contact. The additional electrical contact is configured to electrically connect with an additional device contact of the electronic device.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of this disclosure and together with the description, serve to explain the principles of the disclosure.

FIGS. 1A and 1B are perspective views of an exemplary endoscope, according to aspects of this disclosure.

FIG. 2 depicts a distal portion of an exemplary endoscope, according to aspects of this disclosure.

FIGS. 3A and 3B depict views of an exemplary electronic package for use in an endoscope, according to aspects of this disclosure.

FIG. 4 depicts a side view of an exemplary electronic package for use in an endoscope, according to aspects of this disclosure.

FIG. 5 depicts a perspective view of an exemplary electronic package for use in an endoscope, according to aspects of this disclosure.

FIG. 6 depicts a top view of an exemplary electronic package for use in an endoscope, according to aspects of this disclosure.

FIG. 7 depicts an additional view of an exemplary electronic package for use in an endoscope, according to aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the patient. Throughout the figures included in this application, arrows labeled “P” and “D” are used to show the proximal and distal directions in the figure. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Further, relative terms such as, for example, “about,” “substantially,” “approximately,” etc., are used to indicate a possible variation of +10% in a stated numeric value or range.

Aspects of this disclosure relate to electronic packages for use in medical devices such as endoscopes. As discussed above, forward-facing endoscopes typically have an image sensor mounted such that a plane of the image sensor is orthogonal, or approximately orthogonal, to a long axis of the endoscope. For example, in some applications, an image sensor is mounted on a flexible circuit board, which is bent at or around ninety degrees to the plane of the image sensor. This approach to mounting permits the image sensor to be placed in a distal tip of the endoscope such that the sensor is forward looking and provides a connection for cables that run along the long axis of the endoscope. But such a bend in the circuit board can be difficult to achieve and carries a risk of breaking electrical traces and/or components on the flexible circuit board.

By contrast, disclosed solutions facilitate mounting and electrical connection of an image sensor without posing the above-mentioned risks. For instance, certain aspects relate to electronic packages for image sensors that maintain the plane of the image sensor orthogonal, or approximately orthogonal, to the long axis of the endoscope while being generally oriented along the axis of the endoscope.

In an example, components of an electronic package are positioned proximate to a rear side of the package's image sensor and orthogonal, or approximately orthogonal, to the plane of the image sensor. Electrical connection from the image sensor to other components of the package is made by way of one or more contacts on an end of those components being in contact with one or more device contacts on a rear of the image sensor.

For instance, the electronic package includes various electrical contacts mounted using, for example, surface mount technology (SMT) and positioned at an edge of the electronic package. The placement of the contacts facilitates electrical connection with corresponding electrical contacts that protrude from the electronic device of the package (e.g., the image sensor). Soldering and/or adhesive may be used between the electronic device and other components of the electronic package, as appropriate.

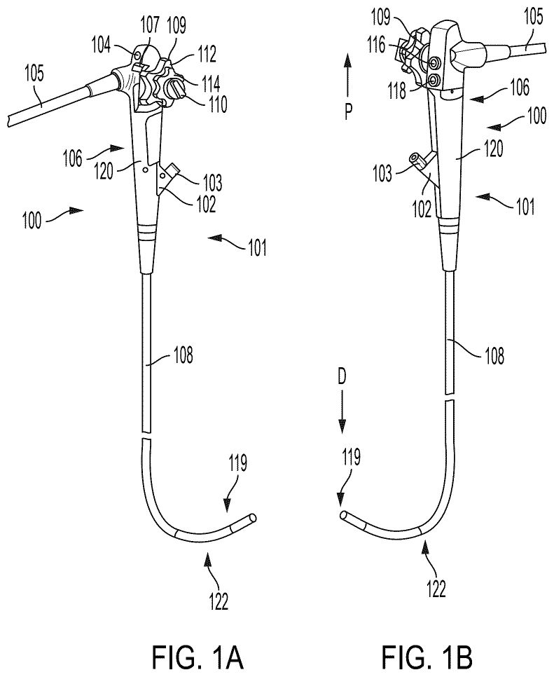

Turning now to the figures, FIGS. 1A and 1B show perspective views of an exemplary endoscope system 100. Endoscope system 100 may include an endoscope 101 and other system components (not shown), such as a controller, a light source, a source of suction and/or irrigation, etc. Endoscope 101 may include a handle assembly 106 and a flexible tubular shaft 108. The handle assembly 106 may include a biopsy port 102, a biopsy cap 103, an image capture button 104, an elevator actuator 107 (if endoscope 101 is a duodenoscope), a first locking lever 109, a second locking lever 110, a first control knob 112, a second control knob 114, a suction button 116, an air/water button 118, a handle body 120, and an umbilicus 105. All of the actuators, elevators, knobs, buttons, levers, ports, or caps of endoscope 101, such as those enumerated above, may serve any purpose and are not limited by any particular use that may be implied by the respective naming of each component used herein. The umbilicus 105 may extend from handle body 120 to one or more auxiliary devices, water/fluid supply, and/or vacuum source. Umbilicus 105 therefore may transmit signals between endoscope 101 and a controller, in order to control lighting and imaging components of endoscope 101 and/or receive image data from endoscope 101. Umbilicus 105 also can provide fluid for irrigation from the water/fluid supply and/or suction to a distal tip 119 of shaft 108. Buttons 116 and 118 include control valves for suction and fluid supply (e.g., air and water), respectively.

Shaft 108 may terminate at distal tip 119. Shaft 108 may include an articulation section 122 for deflecting distal tip 119 in up, down, left, and/or right directions. Knobs 112 and 114 may be used for controlling such deflection, and locking levers 109 and 110 may lock knobs 112 and 114, respectively, in desired positions. Handle body 120 may be tapered and may narrow as the handle extends distally such that the profile of handle body 120 is smaller at its distal end than at its proximal end.

In operating endoscope system 100, a user may use their left hand to hold handle assembly 106 while the right hand is used to hold accessory devices and/or operate one or more of the actuators of handle assembly 106, such as first and second control knobs 112, 114 and first and second locking levers 109, 110. The user may grasp the handle assembly 106 by wrapping the user's hand around handle body 120. When grasping handle body 120, the user may use the left thumb to operate first and second control knobs 112, 114 and the elevator actuator 107 (through rotation about their respective axes), and may use a left-hand finger to operate the image capture button 104, the suction button 116, and/or the air/water button 118 (each by pressing). The user may rotate the handle assembly 106 (e.g., by moving his/her wrist) in order to rotate shaft 108 about a longitudinal axis of shaft 108 and position distal tip 119 at a target area within a patient's body.

The user may actuate button 104 to initiate video display from the imaging device, take an image (such as a digital image) with the imaging device, and/or take any other action associated with the imaging device. During operation, the user may visualize the video feed from the imaging device on an electronic display. The real-time signal from the imaging device may be processed by a control unit and output to the electronic display. The electronic display may be one or more electronic displays such as, for example, a monitor, television, tablet, smartphone, portion of a control unit, virtual reality display, or other display device.

Although the term endoscope may be used herein, it will be appreciated that other devices, including, but not limited to, other types of scopes, endoscopes, and medical devices, such as cholangioscopes, duodenoscopes, colonoscopes, ureteroscopes, bronchoscopes, laparoscopes, sheaths, catheters, or any other suitable delivery device or medical device, may be used in connection with the devices of this disclosure, and the devices, systems, and methods discussed below may be incorporated into any of these or other medical devices.

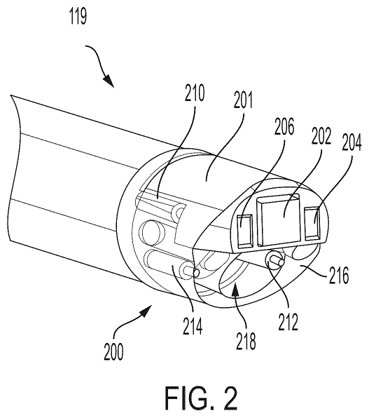

FIG. 2 depicts a distal portion of an exemplary endoscope, according to aspects of this disclosure. End 200 forms part of distal tip 119. End 200 includes a cap 201; an imaging sensor 202; a lighting source 204; a lighting source 206; cables 210; wires 212 and 214; an overmold 216; and a distal opening 218 of a working channel. Additional components are possible.

Cap 201 curves around and contains some of the components of end 200 and protects the components from damage. Those components include imaging sensor 202 and lighting sources 204 and 206, which sit in apertures defined by cap 201. In some aspects, cap 201 can also provide electro-magnetic shielding for the components.

Imaging sensor 202 is positioned such that the plane of the imaging sensor is orthogonal, or approximately orthogonal, to a longitudinal axis of shaft 108. Examples of imaging sensor 202 include image sensors such as charge coupled device (CCD) sensors and complementary metal-oxide semiconductor (CMOS) sensors.

Imaging sensor 202 may include one or more lenses. Lighting sources 204 and 206 are positioned on either side of lighting source, but may be positioned differently in some embodiments. In some embodiments, only one lighting source may be used. Examples of lighting sources 204 and 206 include Light Emitting Diodes (LEDs).

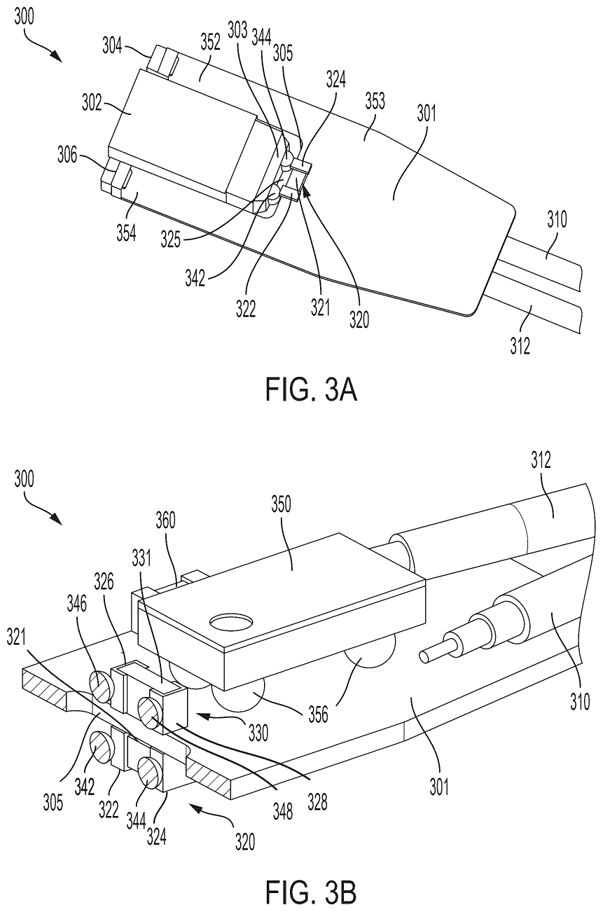

Cables 210 extend through a lumen of endoscope 101 to distal tip 119. Cables 210 connect one or more components such as the imaging sensor 202, lighting sources 204 and 206, and/or other components to a control unit (not depicted), often via additional electrical components in handle 106 and umbilicus 105. Distal tip 119 can include an electronic package that includes imaging sensor 202, as discussed further with respect to FIGS. 3A-8.

Wires 212 and 214 are two of four steering wires used for controlling steering of distal tip 119. The other two wires are blocked from view. Wires 212 and 214 extend through a lumen of endoscope 101 from handle 106 to distal tip 119. Overmold 216 may be a resin molded over various components in distal tip 119 (such as wires 212, 214 and cables 210), to secure those components in place. Overmold 216 also defines distal opening 218 of a working channel. Distal opening 218 is in communication with port 102 and receives, for example, tools or instruments for performing a medical procedure.

FIGS. 3A and 3B each depict a view of an exemplary electronic package 300 for use in an endoscope, according to aspects of this disclosure. FIG. 3A depicts a perspective view of a top side of electronic package 300, whereas FIG. 3B represents a perspective view of a bottom side of electronic package 300 with some components shown in cross-section.

Electronic package 300 includes a printed circuit board (PCB) 301, an imaging sensor 302, lighting sources 304 and 306, and cables 310 and 312. As depicted, within electronic package 300, its various components electrically connect with one another, for example, PCB 301 electrically connects with imaging sensor 302 and lighting sources 304, 306, and cables 310, 312 electrically connect with PCB 301. For example, one or more electrical traces, formed of metal such as copper, for connecting electronic components may be placed on PCB 301. Electronic package 300 can be positioned in distal tip 119, and specifically within portions of cap 201 and overmold 216, which can provide structural support, protection, and electrical isolation.

PCB 301 may have arms 352 and 354 and a main body 353 at the base of arms 352, 354. Arms 352 and 354 define a space therebetween for accommodating imaging sensor 302 and can thereby provide structural support for imaging sensor 302 and/or additional electronic devices.

PCB 301 can be made of a rigid material or a flexible material. For example, PCB 301 can be formed of a combination of resin and glass. Examples of flexible substrate material include polyimide. Other materials are possible. PCB 301, including arms 352, 354 and main body 353, can be formed from, or cut from, a single piece of PCB material. In an aspect, PCB 301 may be made of flexible material and may be folded to provide two surfaces for component mounting or additional contacts.

Main body 353 has an edge surface 305, which is perpendicular, or approximately perpendicular, to the top and bottom planar surfaces of PCB 301. Edge surface 305 of PCB 301 is a distally-facing edge surface between a top planar surface of PCB 301 and a bottom (underneath) planar surface of PCB 301. Edge surface 305 may be planar and extend between arms 352, 354 at the proximalmost ends of arms 352, 354. In an aspect, edge surface 305 does not include any electrical traces or electrical contacts.

PCB 301 may connect with one or more mounts, which may include one or more mount components and/or contacts 322, 324, 326, and/or 328. For example, mount 320 is positioned on a top planar surface of PCB 301 at or near edge surface 305. Mount 320 includes a mount component 321 on which contacts 322 and 324 are mounted. Contacts 322 and 324 may be positioned on an exterior surface of mount component 321. Mount 330 is placed on the rear (underneath) surface of PCB 301. Mount 330 includes a mount component 331 on which contacts 326 and 328 are mounted. Contacts 326 and 328 may be positioned on an exterior surface of mount component 331.

In some aspects, contacts 322, 324, 326 and/or 328 may be metal pins that are affixed to the top and/or bottom planar surfaces of PCB 301. Each pin may be soldered to those surfaces and extend distally beyond surface 305, so that ends of the pins electrically connect to contacts 342, 344, 346, and/or 348. In this case, opposite ends of the metal pins electrically connect to respective traces on PCB 301.

In some aspects, mount components 321 and/or 331 may be rotated in-plane of the surface of 301 and aligned lengthwise with an axis of the endoscope. In this case, mount components 321 and/or 331 may be zero ohm resistors.

Imaging sensor 302 is positioned with respect to PCB 301 such that a plane of imaging sensor 302 is orthogonal, or approximately orthogonal, to the top and bottom planar surfaces of PCB 301. The long dimension of PCB 301, and the top an bottom planar surfaces of PCB 301, are generally aligned with the axis of the endoscope. Imaging sensor 302 has a rear surface 303, on which device contacts 342, 344, 346, and 348 are mounted.

As explained below, contacts 322, 324, 326, and 328, which are mounted on PCB 301 can electrically connect with device contacts 342, 344, 346, and 348. Device contacts 342, 344, 346, and 348; contacts 322, 324, 326, and 328, mounts 320 and 330; and/or mount components 321 and 331 or any combination thereof, can provide mounting surfaces for the image sensor and in some cases can support a weight of the image sensor.

The contacts of PCB 301 may align and connect electrically with the device contacts of imaging sensor 302. Contact 322 may connect electrically and align with device contact 342. Contact 324 may connect electrically and align with device contact 344. Contact 326 may connect electrically and align with device contact 346. Contact 328 may connect electrically and align with device contact 348.

As can be seen, the edge surface 305 does not intersect with a plane or grid defined by, or otherwise contact, the device contacts 342, 344, 346, and 348, forming a space 325 between surface 303 and edge surface 305 and between device contacts 342, 344, 346, and 348 and edge surface 305. Different spacing of contacts 342, 344, 346, and 348 are possible. For example, one or more of contacts 342, 344, 346, and/or 348 may protrude slightly over edge surface 305. In other aspects, a proximalmost part of one or more of contacts 342, 344, 346, and/or 348 may be aligned with edge surface 305, or recessed from edge surface 305. Hence, a size of space 325, including a distance between edge surface 305 and surface 303, may be adjusted as appropriate.

Mount component 321 is positioned on PCB 301 so that the distalmost, distal facing surface of mount component 321 is aligned with, and flush with, edge surface 305. But in some aspects, the distalmost, distal facing surface of mount component 321 can be recessed proximally away from edge surface 305 or extended distally from edge surface 305.

Similarly, mount component 331 is positioned on PCB 301 so that the distalmost, distal facing surface of mount component 331 is aligned with, and flush with, edge surface 305. But in some aspects, the distalmost, distal facing surface of mount component 331 can be recessed proximally away from edge surface 305 or extended distally from edge surface 305. Mount components 321 and 331 can be an electrical insulator such as rubber, ceramic or plastic. Other materials are possible.

As depicted, contacts 322 and 324 protrude distally past edge surface 305. For example, if mount component 321 is aligned with edge surface 305, then contacts 322 and/or 324 may protrude from edge surface 305. But in some aspects, contacts 322 and 324 can be recessed proximally from edge surface 305 or align with, and be flush with, edge surface 305. Additionally or alternatively, contacts 322 and 324 can each be mounted directly on PCB 301.

Contacts 322, 324, 326, and 328 are each formed of an electrically conductive material, are spaced from each other, and have a respective surface. Each surface may be distally-facing, proximally-facing, and/or side-facing. For example, as depicted, contacts 322 and 324 each wrap around a respective side of mount component 321 and contacts 326 and 328 each wrap around a respective side of mount component 331. But in some cases, the contacts may be positioned only on a single surface of the mount components.

FIG. 3B depicts a cross section of a part of electronic package 300. Arms 352 and 354 are not depicted. FIG. 3B depicts a cross-section view of device contacts 342, 344, 346, and 348, which are depicted in a hatched shading.

As can be seen, device contacts 342 and 344 are positioned on the top planar surface of PCB 301 (the top surface being the surface shown in FIG. 3A) and are in contact with contacts 322 and 324, while contacts 346 and 348 are positioned below the bottom planar surface of PCB 301 and are in contact with contacts 326 and 328. Size, spacing, and position of contacts 342, 344, 346, and 348, and/or contacts 322, 324, 326, and 328, can be adjusted as appropriate to facilitate mounting of different imaging sensors.

While contacts 342, 344, 346, and 348 form a 2×2 Ball Grid Array (BGA), any number of contacts is possible. Examples of suitable numbers of contacts in a BGA include two, four, six, eight, and so forth. For example, additional contacts can be provided in a stacked manner with additional PCBs placed parallel to and on top of or below PCB 301.

Imaging sensors having different BGA configurations can be accommodated. For example, a BGA can have a different contact spacing or pitch as compared to contacts 342, 344, 346, and 348. For instance, if the BGA ball pitch is not similar to the pitch of the components providing a mounting surface, then such components can attach to one contact only and zero ohm resistors can be used to provide additional connections to PCB 301.

Mount components 321 and/or 331 can be an electrical insulator such as rubber, ceramic, or plastic. But in some aspects, mount components 321 and 331 may be formed from electronic components such as a resistor with high resistance or a capacitor. For instance, mount component 321 can be a capacitor, which can serve an additional purpose of providing power supply decoupling and enhancing the performance of image sensor 302.

Components, conductive traces, and/or cables may be mounted the top planar surface, the bottom planar surface, or both. The traces may connect cables 310 and/or 312 to various components mounted on the PCB such as electronic component 350; contacts 322, 324, 326, and/or 328; and/or lighting sources 304 and/or 306.

Lighting sources 304 and 306 can be used to illuminate objects for viewing by imaging sensor 302. Different wavelengths of light may be used. For instance, lighting source 304 may emit a different range of wavelengths as compared to lighting source 306.

In some aspects, electronic package 300 supports or includes one or more lighting sources. For example, lighting source 304 is fixed to a distal end of arm 352, and lighting source 306 is fixed to a distal end of arm 354. While lighting sources are depicted, arms 352 and 354 can facilitate mounting of and provide support for other electronic devices such as additional image sensors, or other electronic components such as gyroscopes, position sensors, depth sensors, pressure sensors, temperature sensors, and so forth. In some aspects, arms 352 and 354 are not included. In yet other aspects, only one of arm 352 and 354 is present.

Electrical connections may be made to lighting sources 304 and 306, for example, via electrical traces on PCB 301 (including the respective arms 352, 354) and/or dedicated wiring. In some aspects, zero-resistance components can be used to connect lighting sources 304 and 306 to PCB 301. In some aspects, lighting sources 304 and 306 can be connected to PCB 301 via contacts (e.g., similar to contacts 322 and 324). Such contacts may be soldered to PCB 301. The contacts can in turn connect to one or more electrical traces (not shown) and/or cables such as a twisted pair or coaxial cable. In an example, each lighting source can require an anode and a cathode connection. In some aspects, a common cathode connection can be shared between lighting sources.

In some aspects, lighting sources 304 and 306 can share one or more electrical connections from imaging sensor 302. For instance, lighting sources 304 and 306 can share one or more power connections with imaging sensor 302. In some embodiments, lighting sources 304 and 306 may not be present. In other embodiments, multiple imaging sensors may be accommodated.

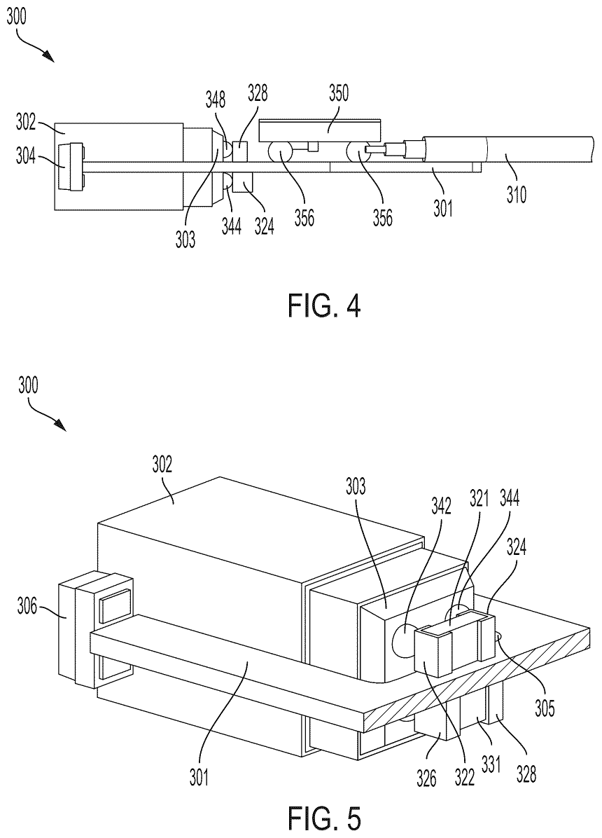

In some aspects, one or more conductor traces can be mounted on PCB 301 such that additional components may be connected at a later time. Such traces may be connected to ground, power, or other signals. Additional components such as resistors, capacitors, diodes, integrated circuits, etc., may be soldered and/or adhered to the surface of PCB 301. In this manner, multiple components can be mounted orthogonally to the plane (to the top and/or bottom planar surfaces) of PCB 301. These components can be mounted on, affixed to, and/or soldered to PCB 301. Additional components are discussed further with respect to FIGS. 4, 5, 6, and 7.

In some aspects, additional contacts are provided on PCB 301 for attaching cables, such as cables 310 and 312. Cables 310 and 312 can carry one or more electrical signals from the imaging sensor 302 to a controller and/or electrical power or other signals, including control signals, to imaging sensor 302 and/or lighting sources 304 and 306.

Contacts 322 and 324 may be soldered to PCB 301. Standard surface-mounting techniques (SMT) can be used. In an example, a solder having a first melting point is used to solder contacts 322 and 324 to PCB 301. Then, a second solder having a second melting point that is lower than the first melting point is used to solder device contacts 342 and 344 to contacts 322 and 324. In some cases, a conductive silver epoxy is used to affix the contacts to PCB 301.

In some aspects, the electronic components are backfilled with epoxy such that when the imaging sensor 302 is attached, even if any solder of the surface mounted components were to reflow, the epoxy would hold the already-mounted components in place.

Standard pick and place equipment and SMT reflow can be used. After component attachment (with or without epoxy), the PCBs can be individualized from the array and placed into a holder that can hold the PCB perpendicular to the ground such that the mounting surface provided by the SMT components is parallel to the ground. Then, imaging sensor 302 can be placed and soldered using standard pick and place and SMT reflow equipment.

FIG. 3B and FIG. 4 (side view) depict exemplary electronic package 300 with additional optional components. Specifically, package 300 further includes a plurality of component contacts 356 (in this case four) and electronic components 350 and 360. Electronic component 350 may be soldered to PCB 301 via component contacts 356. Electronic component 360 may be similarly attached to PCB 301. Component contacts 356 may be connected to cables 310 and/or 312 by way of an electrical trace and/or additional contacts. For example, cables 310 and/or 312 may each have a respective contact on PCB 301 and then be connected to the components via one or more electrical traces. Examples of electronic components 350 and 360 include oscillators, integrated circuits, processors, and so forth.

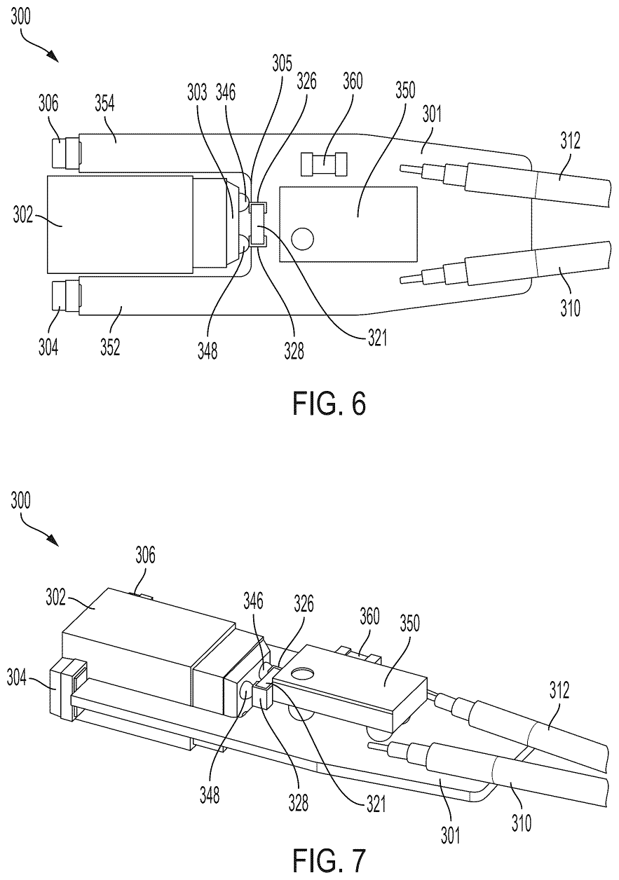

FIG. 5 depicts a perspective cross-sectional view of exemplary electronic package 300, FIG. 6 depicts a view of the bottom planar surface of electronic package 300, and FIG. 7 depicts an additional perspective view of electronic package 300.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may, at times, be communicated through the Internet or various other telecommunication networks. Such communications, e.g., may enable loading of the software from one computer or processor into another. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. An electronic package for a medical device, the package comprising:
an electronic device with a rear device surface having at least one device electrical contact;
a printed circuit board (PCB) having (i) a PCB surface with one or more electrical traces and (ii) an edge surface transverse to the PCB surface, wherein the edge surface faces and is spaced from the rear device surface; and
an electrical component fixed to the PCB surface and electrically connected with the at least one device electrical contact and the one or more electrical traces.

2. The electronic package of claim 1, wherein the electrical component is mounted flush with the edge surface.

3. The electronic package of claim 1, wherein the electrical component protrudes distally away from the edge surface.

4. The electronic package of claim 1, wherein the electrical component is mounted recessed from the edge surface.

5. The electronic package of claim 1, wherein the at least one device electrical contact protrudes from the rear device surface.

6. The electronic package of claim 5, wherein the printed circuit board is spaced from the at least one device electrical contact.

7. The electronic package of claim 1, wherein the electrical component comprises at least one contact that is electrically connected with the at least one device electrical contact.

8. The electronic package of claim 6, wherein the printed circuit board further comprises a bottom surface, an additional electrical component positioned on the bottom surface, and an additional contact positioned on the additional electrical component.

9. The electronic package of claim 8, wherein the additional electrical component is electrically connected to the electronic device.

10. The electronic package of claim 1, wherein the electronic device further comprises a ball grid array (BGA) that includes the at least one device electrical contact.

11. The electronic package of claim 1, wherein the electrical component comprises at least one electrical contact and wherein the electronic device is an imaging sensor that is electrically connected, via the at least one device electrical contact and the at least one electrical contact, to the one or more electrical traces.

12. The electronic package of claim 1, wherein the edge surface does not include any electrical traces or electrical contacts.

13. The electronic package of claim 1, wherein the printed circuit board comprises:
a first arm and a second arm, wherein the first arm and the second arm extend distally from a base of the printed circuit board, and wherein the electronic device is positioned between the first arm and the second arm and distally of the base; and
an illumination source positioned at an end of either the first arm or the second arm.

14. The electronic package of claim 1, wherein the electrical component includes an insulator, a first component electrical contact, and a second component electrical contact spaced and insulated from the first component electrical contact, and the at least one device electrical contact includes a first device electrical contact and a second device electrical contact spaced and insulated from the first device electrical contact, and wherein the first component electrical contact touches the first device electrical contact, and the second component electrical contact touches the second device electrical contact.

15. The electronic package of claim 1, wherein the electrical component is a resistor or a capacitor.

16. An endoscope comprising:
an imaging sensor comprising a rear device surface having a plurality of device electrical contacts, the imaging sensor positioned in a distal end of the endoscope;
a printed circuit board (PCB) having (1) a PCB surface and (2) an edge surface transverse to the PCB surface, wherein the edge surface faces the rear device surface and is spaced from the rear device surface;
an insulator affixed to the PCB surface; and
a plurality of electrical contacts affixed to the insulator and electrically connected to the plurality of device electrical contacts.

17. The endoscope of claim 16, wherein the printed circuit board comprises a first arm and a second arm, and wherein the imaging sensor is positioned between the first arm and the second arm, the endoscope further comprising an illumination source positioned on either the first arm or the second arm.

18. The endoscope of claim 16, wherein the printed circuit board further comprises one or more electrical traces, and wherein the imaging sensor is electrically connected, via the plurality of device electrical contacts and the plurality of electrical contacts, to the one or more electrical traces.

19. A connector for an electronic device, the connector comprising:
a first arm;
a second arm, wherein the first arm and the second arm define a space therebetween configured to receive the electronic device, and
a body portion at a base of the first arm and the second arm, the body portion comprising:
a top planar surface with electrical traces;
a bottom planar surface;
an edge surface connecting the top planar surface and the bottom planar surface and facing the space; and
an electrical contact that is disposed on the top planar surface, wherein a contact surface of the electrical contact is either aligned with the edge surface or is above the space, and wherein the electrical contact is configured to electrically connect with a device contact of the electronic device.

20. The connector of claim 19, wherein the bottom planar surface comprises an additional electrical contact, and wherein the additional electrical contact is configured to electrically connect with an additional device contact of the electronic device.
